(19) United States
(12) Patent Application Publication
(10) Pub. No.: US 2026/0209142 A1
(43) Pub. Date: Jul. 23, 2026

(54) PROCESS AND SYSTEM FOR PRODUCING ETHYLENE FROM PYROLYSIS OIL

(71) Applicant: NOVA CHEMICALS (INTERNATIONAL) S.A., Fribourg (CH)

(72) Inventors: Vasily SIMANZHENKOV, Calgary (CA); Abolfazl NOORJAHAN, Calgary (CA); Ce YANG, Clarksville, MD (US); Shahin GOODARZNIA, Calgary (CA); Mohanned MOHAMEDALI, Calgary (CA)

(73) Assignee: NOVA CHEMICALS (INTERNATIONAL) S.A., Fribourg (CH)

(21) Appl. No.: 19/137,754

(22) PCT Filed: Nov. 14, 2023
(86) PCT No.: PCT/IB2023/061511

§ 371 Date: Jun. 11, 2025

Related U.S. Application Data
(60) Provisional application No. 63/432,545, filed on Dec. 14, 2022.

(51) Int. Cl. C07C 4/06; B01J 8/04; B01J 23/44; B01J 29/06; C10G 1/02

(52) U.S. Cl. CPC C07C 4/06; B01J 8/0492; B01J 23/44; B01J 29/06; C10G 1/02; C10G 2300/1003; C10G 2400/20

(57) ABSTRACT

A process for producing ethylene includes contacting a feed stream including pyrolysis oil with a hydrocracking catalyst disposed within a hydrocracking unit to convert, in the presence of hydrogen, at least a portion of the pyrolysis oil into one or more C2-C4 alkanes to produce a yield in a range of from about 40 wt. % to about 100 wt. % of the one or more C2-C4 alkanes. A hydrocracking product stream is removed from the hydrocracking unit. The hydrocracking product stream is diluted with steam to form a steam cracking feed stream. The steam cracking feed stream is heated in a steam cracker to convert at least a portion of the one or more C2-C4 alkanes to ethylene.

### TECHNICAL FIELD

The present disclosure relates to the field of ethylene production. In particular, the present disclosure is directed to systems and processes for producing ethylene from pyrolysis oil using hydrocracking integrated with steam cracking.

### BACKGROUND ART

Pyrolysis is the process of thermally decomposing a material at elevated temperatures in an inert atmosphere and involves a change in chemical composition of the material. Pyrolysis can be performed on a material that is typically regarded as waste (such as biomass or waste plastic) into useful compounds, such as various forms of carbon, syngas, and biochar. For example, pyrolysis can be performed on waste plastic to convert the waste plastic into a usable oil. In some cases, the oil produced by pyrolysis is further processed to convert the oil into other products.

### SUMMARY OF INVENTION

Certain aspects of the subject matter can be implemented as a process for producing ethylene. A feed stream includes pyrolysis oil. The feed stream is contacted with a hydrocracking catalyst disposed within a hydrocracking unit to convert, in the presence of hydrogen, at least a portion of the pyrolysis oil into one or more C2-C4 alkanes to produce a yield in a range of from about 40 wt. % to about 100 wt. % of the one or more C2-C4 alkanes. A hydrocracking product stream is removed from the hydrocracking unit. The hydrocracking product stream includes the one or more C2-C4 alkanes. The hydrocracking product stream is diluted with steam to form a steam cracking feed stream. The steam cracking feed stream is heated in a steam cracker to convert at least a portion of the one or more C2-C4 alkanes to ethylene.

This, and other aspects, can include one or more of the following features. The pyrolysis oil can be derived from pyrolysis of waste plastic. The hydrocracking product stream can include at least 90 wt. % of the one or more C2-C4 alkanes. The hydrocracking product stream can include at least 95 wt. % of the one or more C2-C4 alkanes. The pyrolysis oil can include from about 20 wt. % to about 40 wt. % of one or more linear alkanes (paraffins). The pyrolysis oil can include from 0 wt. % to about 40 wt. % of one or more cyclic alkanes (naphthenes). The pyrolysis oil can include from 0 wt. % to about 50 wt. % of one or more alkenes (olefins). The pyrolysis oil can include from 0 wt. % to about 40 wt. % of one or more aromatic compounds. The pyrolysis oil can include from 0 wt. % to about 50 wt. % of one or more C15+ hydrocarbons. The hydrocracking catalyst can include natural zeolites, synthetic zeolites, bauxite, alkali oxides, alkaline metal earth oxides, aluminum phosphates, transition metal oxides, or any combination of these. The hydrocracking catalyst can include palladium dispersed on a zeolite support. Converting the portion of the one or more C2-C4 alkanes to ethylene can produce hydrogen. The process can include separating at least a portion of the hydrogen from the ethylene and recycling the separated portion of the hydrogen to the hydrocracking unit. Removing the hydrocracking product stream from the hydrocracking unit can include separating hydrogen and methane from the one or more C2-C4 alkanes, such that the hydrocracking product stream is substantially free of hydrogen and methane. The separated hydrogen, the separated methane, or both can be recycled to the hydrocracking unit. Removing the hydrocracking product stream from the hydrocracking unit can include separating C4+ hydrocarbons from the one or more C2-C4 alkanes, such that the hydrocracking product stream is substantially free of C4+ hydrocarbons. At least a portion of the separated C4+ hydrocarbons can be recycled to the hydrocracking unit. Removing the hydrocracking product stream from the hydrocracking unit can include separating at least a portion of propane and butane from the one or more C2-C4 alkanes, such that the hydrocracking product stream includes less than about 7 wt. % of propane and less than about 4 wt. % of butane. The process can include adding ethane to the hydrocracking product stream prior to dilution with steam, such that the hydrocracking product stream includes less than about 7 wt. % of propane and less than about 4 wt. % of butane. The process can include purifying the feed stream to remove heteroatom-containing compounds, metal-containing compounds, or any combinations of these from the feed stream prior to contacting the feed stream with the hydrocracking catalyst in the presence of hydrogen. Purifying the feed stream can include contacting the feed stream with a hydrotreatment catalyst in the presence of hydrogen under hydrotreatment conditions and saturating at least a portion of olefins present in the feed stream into naphthenes, paraffins, or any combinations of these. A mass ratio of the ethylene produced by the steam cracker to the pyrolysis oil of the feed stream entering the hydrocracking unit can be in a range of from about 6:10 to about 9:10. The mass ratio of the ethylene produced by the steam cracker to the pyrolysis oil of the feed stream entering the hydrocracking unit can be in a range of from about 7:10 to about 8:10. The mass ratio of ethylene produced by the steam cracker to the pyrolysis oil of the feed stream entering the hydrocracking unit can be about 3:4.

Certain aspects of the subject matter can be implemented as a system for producing ethylene. The system includes a feed stream, a hydrocracking unit, a hydrocracking product stream, and a steam cracker. The feed stream includes pyrolysis oil. The hydrocracking unit includes a hydrocracking catalyst. The hydrocracking catalyst is configured to convert, in the presence of hydrogen, at least a portion of the pyrolysis oil into one or more C2-C4 alkanes in response to contacting the feed stream to produce a yield in a range of from about 40 wt. % to about 100 wt. % of the one or more C2-C4 alkanes. The hydrocracking product stream is from the hydrocracking unit. The hydrocracking product stream includes the one or more C2-C4 alkanes. The steam cracker is configured to receive and heat at least a portion of the hydrocracking product stream diluted with steam to convert at least a portion of the one or more C2-C4 alkanes to ethylene.

This, and other aspects, can include one or more of the following features. The pyrolysis oil can be derived from pyrolysis of waste plastic. The hydrocracking product stream can include at least 90 wt. % of the one or more C2-C4 alkanes. The hydrocracking product stream can include at least 95 wt. % of the one or more C2-C4 alkanes. The pyrolysis oil can include from about 20 wt. % to about 40 wt. % of one or more linear alkanes (paraffins). The pyrolysis oil can include from 0 wt. % to about 40 wt. % of one or more cyclic alkanes (naphthenes). The pyrolysis oil can include from 0 wt. % to about 50 wt. % of one or more alkenes (olefins). The pyrolysis oil can include from 0 wt. % to about 40 wt. % of one or more aromatic compounds. The pyrolysis oil can include from 0 wt. % to about 50 wt. % of one or more C15+ hydrocarbons. The hydrocracking catalyst can include natural zeolites, synthetic zeolites, bauxite, alkali oxides, alkaline metal earth oxides, aluminum phosphates, transition metal oxides, or any combination of these. The hydrocracking catalyst can include palladium dispersed on a zeolite support. The system can include a de-methanizer column. The de-methanizer column can be configured to receive the hydrocracking product stream and separate hydrogen and methane from the one or more C2-C4 alkanes, such that the hydrocracking product stream is substantially free of hydrogen and methane. The hydrocracking unit can be configured to receive the separated hydrogen, the separated methane, or both. The system can include a separator that is configured to receive the hydrocracking product stream and separate C4+ hydrocarbons from the one or more C2-C4 alkanes, such that the hydrocracking product stream is substantially free of C4+ hydrocarbons. The hydrocracking unit can be configured to receive at least a portion of the separated C4+ hydrocarbons. The system can include a de-ethanizer column. The de-ethanizer column can be configured to receive the hydrocracking product stream and remove ethane to form an ethane stream. The ethane stream can be diluted with steam and heated by the steam cracker. The system can include an ethane stream that mixes with the hydrocracking product stream upstream of the steam cracker, such that the portion of the hydrocracking product stream that is diluted with steam and heated by the steam cracker includes less than about 7 wt. % of propane and less than about 4 wt. % of butane. The system can include a purification unit upstream of the hydrocracking unit. The purification unit can be configured to remove heteroatom-containing compounds, metal-containing compounds, or any combinations of these from the feed stream. The purification unit can include a hydrotreater. The hydrotreater can include a hydrotreatment catalyst. The hydrotreatment catalyst can be configured to, in response to contacting the feed stream in the presence of hydrogen under hydrotreatment conditions, saturate at least a portion of olefins present in the feed stream into naphthenes, paraffins, or any combinations of these. A mass ratio of the ethylene produced by the steam cracker to the pyrolysis oil of the feed stream entering the hydrocracking unit can be in a range of from about 6:10 to about 9:10. The mass ratio of the ethylene produced by the steam cracker to the pyrolysis oil of the feed stream entering the hydrocracking unit can be in a range of from about 7:10 to about 8:10. The mass ratio of ethylene produced by the steam cracker to the pyrolysis oil of the feed stream entering the hydrocracking unit can be about 3:4.

### BRIEF DESCRIPTION OF DRAWINGS

FIG. 1 is a block diagram of an example system for producing ethylene.

FIG. 2 is a block diagram of an example system for producing ethylene that includes ethane addition prior to steam cracking.

FIG. 3 is a block diagram of an example system for producing ethylene that includes ethane separation prior to steam cracking.

FIG. 4 is a schematic diagram of an example reactor including a catalyst.

FIG. 5 is a schematic diagram of an example distillation column.

FIG. 6 is a schematic diagram of an example steam cracking unit.

FIG. 7 is a flow chart of an example method for producing ethylene.

FIG. 8 is a plot of vaporization percent versus temperature for an example pyrolysis oil.

FIG. 9 is a plot of percent yield for various components versus time on stream for an example hydrocracking process.

### DESCRIPTION OF EMBODIMENTS

This disclosure describes systems and processes for producing ethylene from pyrolysis oil (also referred to as pyoil) using hydrocracking integrated with steam cracking. The pyrolysis oil is derived from the pyrolysis of waste plastic. The pyrolysis oil undergoes a hydrocracking process in the presence of hydrogen for conversion into C2-C4 alkanes. The C2-C4 alkanes then undergo a steam cracking process for conversion into ethylene. Hydrogen can be separated from the ethylene and recycled to the hydrocracking process. The subject matter described can be implemented to realize one or more of the following advantages. While conventional hydrocracking processes utilize crude oil as feedstock, the systems and processes described utilize pyrolysis oil that has been derived from waste plastic. The pyrolysis oil derived from waste plastic can be converted into ethylene at greater yields (for example, greater than 70% conversion by weight into ethylene) in comparison to conventional processes. The hydrocracking processes described herein can convert pyrolysis oil derived from waste plastic into light C1-C4 hydrocarbons. The hydrocracking processes described herein can convert heavier portions of the pyrolysis oil in comparison to conventional hydrocracking process. For example, conventional hydrocracking processes can typically convert lighter portions of the pyrolysis oil, up to about 171 degrees Celsius (° C.) boiling point cut, whereas the hydrocracking processes described herein can convert heavier portions of the pyrolysis oil, up to about 399° C. boiling point cut. Thus, a larger portion (and in some cases, close to 100%) of the pyrolysis oil can be converted by the systems and processes described herein.

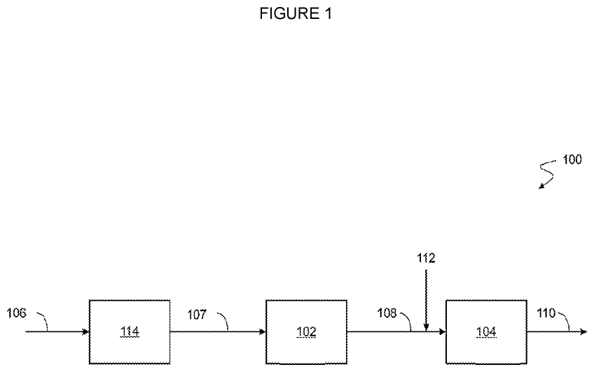

FIG. 1 depicts an example system 100 for producing ethylene. The system 100 includes a hydrocracking unit 102 and a steam cracking unit 104. The system 100 includes a feed stream 107, a hydrocracking product stream 108, and an ethylene product stream 110. The feed stream 107 includes pyrolysis oil that is derived from pyrolysis of synthetic rubber and/or waste plastic, including but not limited to polyesters (for example, polyethylene terephthalate (PET) or polycaprolactone), polyolefins (for example, low-density polyethylene (LDPE), linear low-density polyethylene (LLDPE), medium-density polyethylene (MDPE), high-density polyethylene (HDPE), or polypropylene (PP)), polyvinyl chloride (PVC), polystyrene (PS), polycarbonates, polylactides, polyethers, polyacrylates, acrylonitrile rubbers (for example, acrylonitrile butadiene styrene (ABS), styrene-acrylonitrile resin (SAN), acrylonitrile styrene acrylate (ASA), acrylonitrile butadiene rubber (NBR)), nylons, polyurethanes, or any combination or copolymers of these. Pyrolysis is the process of thermal decomposition of a material (such as waste plastic) at elevated temperatures in an inert atmosphere (for example, absence of oxygen). In some implementations, the pyrolysis oil includes from 0 weight percent (wt. %) to about 20 wt. % of one or more aromatic compounds (such as benzene, xylene, or toluene). For example, the pyrolysis oil includes less than about 15 wt. %, less than about 10 wt. %, less than about 5 wt. %, or less than about 1 wt. % of one or more aromatic compounds. For example, the pyrolysis oil includes from about 1 wt. % to 20 wt. %, from about 1 wt. % to about 15 wt. %, from about 1 wt. % to about 10 wt. %, or from about 1 wt. % to about 5 wt. % of one or more aromatic compounds.

The hydrocracking unit 102 includes a hydrocracking catalyst (for example, disposed within a hydrocracking reactor). The hydrocracking catalyst is configured to convert, in response to contacting the feed stream 107, at least a portion of the pyrolysis oil of the feed stream 107 in the presence of hydrogen into one or more C1-C4 alkanes. C1-C4 alkanes can include alkanes having a number of carbon atoms from 1 to 4. Examples of C1-C4 alkanes include methane (C1), ethane (C2), propane (C3), and butane (C4). C4+ alkanes are alkanes that have a number of carbon atoms greater than 4. In some implementations, the hydrocracking unit 102 is configured to convert at least about 80 wt. % of the pyrolysis oil of the feed stream 107 into one or more C1-C4 alkanes. For example, the hydrocracking unit 102 can be configured to convert from about 80 wt. % to 100 wt. %, from about 90 wt. % to 100 wt. %, or from about 95 wt. % to 100 wt. % of the pyrolysis oil of the feed stream 107 into one or more C1-C4 alkanes. Hydrogen gas can be provided to the hydrocracking reactor along with the feed stream 107. In some implementations, a volume ratio of hydrogen gas to the feed stream 107 (pyrolysis oil) entering the hydrocracking reactor is equal to or less than 2,000:1. The hydrocracking unit 105 can convert a wide range of compositions of the feed stream 107 into C1-C4 alkanes. For example, the hydrocracking unit 105 can convert the feed stream 107 free of (that is, 0%) C15+ hydrocarbon content (considered as a light pyoil) into C1-C4 alkanes. As another example, the hydrocracking unit 105 can convert the feed stream 107 having a C15+ hydrocarbon content of up to about 50% (with 50% considered as a heavy/waxy pyoil) into C1-C4 alkanes. In some implementations, the hydrocracking unit 102 is configured to convert the feed stream 107 to produce a yield in a range of from about 40 wt. % to about 100 wt. %, from about 60 wt. % to about 98 wt. %, or from about 70 wt. % to about 95 wt. % of one or more C2-C4 alkanes.

In some implementations, the hydrocracking catalyst includes a metallic component and a support. For example, the metallic component can include palladium (Pd), rhodium (Rh), ruthenium (Ru), iridium (Ir), osmium (Os), copper (Cu), cobalt (Co), nickel (Ni), platinum (Pt), iron (Fe), zinc (Zn), gallium (Ga), indium (In), molybdenum (Mo), tungsten (W), vanadium (V), or any combination of these. For example, the support can include a naturally occurring zeolite that is mined or synthetically manufactured (such as mordenite, cancrinite, gmelinite, faujasite, or clinoptilolite) or a man-made zeolite (such as a synthetic zeolite), or any of their acidic forms.

In some implementations, a reaction temperature maintained in the hydrocracking reactor is in a range of from about 250° C. to about 500° C., from about 300° C. to about 450° C., from about 350° C. to about 410° C., or from about 350° C. to about 450° C. In some implementations, a reactor inlet pressure in the hydrocracking reactor is in a range of from about 3,450 kilopascals gauge (kPag) to about 10,340 kPag or from about 4,825 kPag to about 6,895 kPag. In some implementations, the liquid hourly space velocity (LHSV) of the feed stream 107 in the hydrocracking reactor is in a range of from about 0.2 per hour to about 5 per hour, from about 0.5 per hour to about 3 per hour, from about 0.5 per hour to about 1.25 per hour, or from about 0.75 per hour to about 2 per hour. The hydrocracking product stream 108 is discharged by the hydrocracking unit 102.

The hydrocracking product stream 108 can include from 0 wt. % to about 15 wt. % of hydrogen (H₂). The hydrocracking product stream 108 can include from 0 wt. % to about 10 wt. % of methane. The hydrocracking product stream 108 can include from 0 wt. % to about 20 wt. % of a combined content of hydrogen and methane. The hydrocracking product stream 108 can include from 0 wt. % to about 15 wt. % of ethane. The hydrocracking product stream 108 can include from 0 wt. % to about 45 wt. % of propane. The hydrocracking product stream 108 can include from about 10 wt. % to about 40 wt. % of butane. The hydrocracking product stream 108 can include from about 15 wt. % to about 85 wt. % of LPGs (a combined content of ethane, propane, and butane (and any isomers thereof)). The hydrocracking product stream 108 can include from 0 wt. % to about 70 wt. % of C4+ hydrocarbons. The hydrocracking product stream 108 can be processed to separate components from the hydrocracking product stream 108 prior to being discharged by the hydrocracking unit 102. For example, hydrogen, methane, and C4+ hydrocarbons (that is, hydrocarbons having a number of carbon atoms greater than 4) can be separated from the hydrocracking product stream 108, such that a majority of the hydrocracking product stream 108 is C2-C4 alkanes prior to undergoing cracking in the steam cracking unit 104.

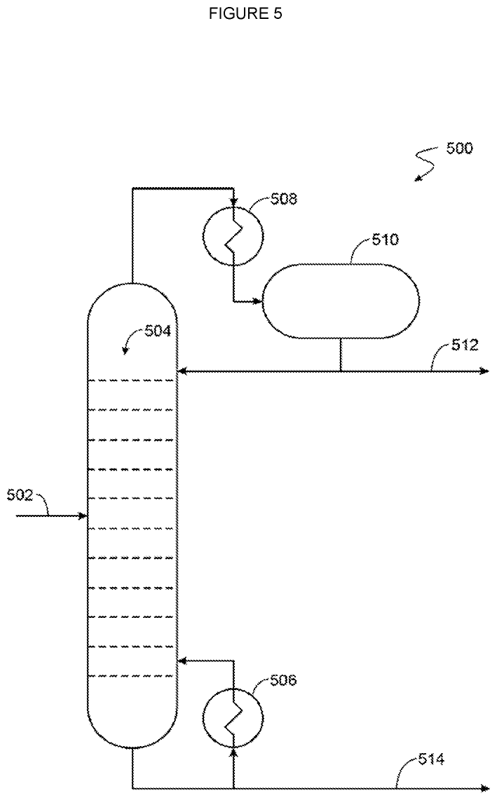

In some implementations, the system 100 includes a de-methanizer column. The de-methanizer column can be configured to receive and fractionate the hydrocracking product stream 108 from the hydrocracking unit 102. The de-methanizer column can be configured to separate hydrogen and methane from a remaining portion of the hydrocracking product stream 108 (such as ethane, propane, and butane). In some implementations, the hydrogen separated by the de-methanizer column is recycled to the hydrocracking unit 102. The remaining portion of the hydrocracking product stream 108 (for example, C2-C4 alkanes) is substantially free of hydrogen and methane and can be flowed to the steam cracking unit 104. For example, the remaining portion of the hydrocracking product stream 108 flowed to the steam cracking unit 104 can have less than about 1 wt. %, less than about 0.5 wt. %, less than about 0.1 wt. %, or less than about 0.01 wt. % of hydrogen. For example, the remaining portion of the hydrocracking product stream 108 flowed to the steam cracking unit 104 can have less than about 1 wt. %, less than about 0.5 wt. %, less than about 0.1 wt. %, or less than about 0.01 wt. % of methane. The separated hydrogen, the separated methane, or both can be recycled to the hydrocracking unit 102. An example of a distillation column which can be implemented as a de-methanizer column is shown in FIG. 5 and described in more detail later.

In some implementations, the system 100 includes a de-ethanizer column. The de-ethanizer column can be configured to receive and fractionate the hydrocracking product stream 108 or the remaining portion of the hydrocracking product stream 108 from the de-methanizer column. The de-ethanizer column can be configured to separate ethane from heavier hydrocarbons, such as propane and butane. The ethane separated from the de-ethanizer column can be flowed to the steam cracking unit 104. An example of a distillation column which can be implemented as a de-ethanizer column is shown in FIG. 5 and described in more detail later.

In some implementations, the system 100 includes a separator. The separator can be configured to receive and fractionate the hydrocracking product stream 108 or the remaining portion of the hydrocracking product stream 108 from the de-methanizer column. The separator can be configured to separate C4+ hydrocarbons from lighter hydrocarbons, such as C2-C4 alkanes. The C2-C4 alkanes can be the hydrocracking product stream 108 that flows to the steam cracking unit 104. The separated C4+ hydrocarbons can be recycled to the hydrocracking unit 102. In some implementations, the separator is a distillation column, and example of which is shown in FIG. 5 and described in more detail later.

The hydrocracking product stream 108 is diluted with steam 112 prior to cracking by the steam cracking unit 104. In some implementations, a mass ratio of steam 112 to the hydrocracking product stream 108 that is provided to the steam cracking unit 104 is in a range of from about 1:5 to about 2:5. In some implementations, a mass ratio of steam 112 to ethane in the hydrocracking product stream 108 that is provided to the steam cracking unit 104 is in a range of from about 1:5 to about 2:5. The steam cracking unit 104 is configured to receive and heat at least a portion of the hydrocracking product stream 108 diluted with steam 112 to convert at least a portion of the one or more C2-C4 alkanes (for example, ethane) to ethylene. The heating in the steam cracking unit 104 is performed in the absence of oxygen, so that combustion of the hydrocarbons in the hydrocracking product stream 108 is avoided. In some implementations, the hydrocracking product stream 108 diluted with steam 112 is heated to a temperature in a range of from about 700° C. to about 1,000° C., from about 800° C. to about 900° C., or from about 800° C. to about 850° C. In some implementations, the operating pressure in the steam cracking unit 104 is in a range of from about 150 kPag to about 200 kPag. In some implementations, the residence time of the hydrocracking product stream 108 diluted with steam 112 through the steam cracker is less than about 1.5 seconds or less than about 1 second. For example, the residence time of the hydrocracking product stream 108 diluted with steam 112 through the steam cracker can be a few milliseconds. The ethylene from the steam cracking unit 104 is separated (for example, by distillation or membrane separation) to form the ethylene product stream 110. In some implementations, the ethylene product stream 110 has an ethylene content of at least 99 wt. % (for example, at least 99.5 wt. %, at least 99.9 wt. %, or at least 99.99 wt. %). In some implementations, a mass ratio of the ethylene product stream 110 to the feed stream 107 (pyrolysis oil) is in a range of from about 6:10 to about 9:10 or from about 7:10 to about 8:10. For example, the mass ratio of the ethylene product stream 110 to the feed stream 107 (pyrolysis oil) is about 3:4. In other words, the system 100 can convert from about 60 wt. % to about 90 wt. %, from about 70 wt. % to about 80 wt. %, or about 75 wt. % of pyrolysis oil (entering the hydrocracking unit 102) into ethylene (exiting the steam cracking unit 104). In some implementations, unreacted ethane from the steam cracking unit 104 is separated and recycled back to the steam cracking unit 104 for conversion to ethylene.

The feed stream 106 may include impurities, depending on the type and composition of the plastic waste used to generate the pyrolysis oil. Some examples of impurities that may exist in the feed stream 106 include compounds including heteroatoms (such as sulfur (S), oxygen (O), nitrogen (N), chlorine (Cl), phosphorus (P)) and metal impurities. Some examples of heteroatom-containing compounds include nitrogen gas (N₂), oxygen gas (O₂), chlorine gas (Cl₂), ammonia (NH₃), and amides. Some examples of metal impurities include compounds including calcium (Ca), magnesium (Mg), iron (Fe), or sodium (Na), which can be bound to hydrocarbon components or exist as parts of other compounds, such as salts (for example, calcium carbonate (CaCO₄), magnesium chloride (MgCl₂), and iron hydroxide (Fe(OH)₃)). As described previously, the types and amount of impurities that exist in the feed stream 106 depend on the type and composition of the feedstock used to generate the pyrolysis oil. Impurities in the feed stream 106 may negatively impact (for example, deactivate) catalyst activity (for example, in the hydrocracking unit 102). Thus, in some cases (and especially in cases where impurities exist in the feed stream 106), it can be beneficial to purify the feed stream 106 (for example, to remove such impurities).

In some implementations, as shown in FIG. 1, the system 100 includes a purification unit 114 upstream of the hydrocracking unit 102. The purification unit 114 is configured to remove impurities (such as heteroatom-containing compounds and/or metal impurities) from the feed stream 106. The purification unit 114 can also saturate olefins in the feed stream 106 into naphthenes and/or paraffins. The purification unit 114 can implement various processes (such as hydrotreatment, adsorption, and absorption) to purify the feed stream 106. In some implementations, the purification unit 114 includes a hydrotreater that includes a hydrotreatment catalyst. The hydrotreatment catalyst can include, for example, nickel-molybdenum (NiMo), nickel-tungsten (NiW), or cobalt-molybdenum (CoMo). In some implementations, the hydrotreatment catalyst is supported by an alumina carrier. As an example, the purification unit 114 can include two packed bed reactors in series, each loaded with hydrotreatment catalyst. The packed bed reactors can be operated at a hydrotreatment temperature in a range of from about 300° C. to about 400° C. or from about 350° C. to about 380° C. For example, the first packed bed reactor can operate at a first hydrotreatment temperature of about 350° C., and the second packed bed reactor can operate at a second hydrotreatment temperature of about 380° C. In some implementations, both packed bed reactors operate at an operating pressure of about 6,895 kPag. In some implementations, the LHSV of the feed stream 106 in each of the packed bed reactors is about 0.5 per hour. Hydrogen gas can be provided to the purification unit 114 along with the feed stream 106. In some implementations, a volume ratio of hydrogen gas to the feed stream 106 entering the purification unit 114 is equal to or less than 2,000:1. For example, the volume ratio of hydrogen gas to the feed stream 106 entering the purification unit 114 is in a range of from about 500:1 to about 2,000:1. In some implementations, the purification unit 114 is configured to remove enough impurities from the feed stream 106, such that the feed stream 107 exiting the purification unit 114 and entering the hydrocracking unit 102 has a sulfur content of less than about 100 parts per million (ppm), less than about 50 ppm, less than about 10 ppm, less than about 1 ppm, or less than about 0.1 ppm. In some implementations, the feed stream 107 exiting the purification unit 114 and entering the hydrocracking unit 102 has a nitrogen content of less than about 100 ppm, less than about 50 ppm, less than about 10 ppm, less than about 1 ppm, or less than about 0.1 ppm. In some implementations, the feed stream 107 exiting the purification unit 114 and entering the hydrocracking unit 102 has an oxygen content of less than about 500 ppm, less than about 200 ppm, less than about 100 ppm, less than about 10 ppm, less than about 1 ppm, or less than about 0.1 ppm. In some implementations, the feed stream 107 exiting the purification unit 114 and entering the hydrocracking unit 102 has a chlorine content of less than about 10 ppm, less than about 5 ppm, less than about 3 ppm, less than about 2 ppm, less than about 1 ppm, or less than about 0.1 ppm. In some implementations, the feed stream 107 exiting the purification unit 114 and entering the hydrocracking unit 102 has a phosphorus content of less than about 2 ppm, less than about 1 ppm, or less than about 0.1 ppm. In some implementations, the feed stream 107 exiting the purification unit 114 and entering the hydrocracking unit 102 has a calcium content of less than about 20 ppm, less than about 10 ppm, less than about 1 ppm, or less than about 0.1 ppm. In some implementations, the feed stream 107 exiting the purification unit 114 and entering the hydrocracking unit 102 has a sodium content of less than about 20 ppm, less than about 10 ppm, less than about 1 ppm, or less than about 0.1 ppm. In some implementations, the feed stream 107 exiting the purification unit 114 and entering the hydrocracking unit 102 has a silicon content of less than about 20 ppm, less than about 10 ppm, less than about 1 ppm, or less than about 0.1 ppm. In some implementations, the feed stream 107 exiting the purification unit 114 and entering the hydrocracking unit 102 has an iron content of less than about 10 ppm, less than about 5 ppm, less than about 3 ppm, less than about 2 ppm, less than about 1 ppm, less than about 0.1 ppm, less than about 0.01 ppm, or less than about 0.001 ppm.

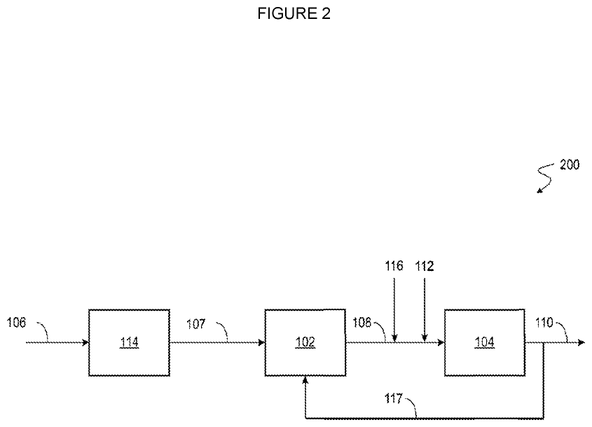

FIG. 2 depicts an example system 200 for producing ethylene that includes ethane addition prior to steam cracking. The system 200 can be substantially similar to the system 100 shown in FIG. 1. In addition to the components described with respect to system 100, system 200 can also include an ethane stream 116 that mixes with the hydrocracking product stream 108 upstream of the steam cracking unit 104. The addition of ethane to the hydrocracking product stream 108 (for example, by mixing with the ethane stream 116) can be implemented in cases where the propane content and/or the butane content of the hydrocracking product stream 108 exceeds the desired level(s) for targeting the production of ethylene in the steam cracking unit 104. In some implementations, it may be desirable to limit the propane content of the hydrocracking product stream 108 to be less than about 7 wt. % for the production of ethylene. In some implementations, it may be desirable to limit the butane content of the hydrocracking product stream 108 to be less than about 4 wt. % for the production of ethylene. For example, in cases where the hydrocracking product stream 108 includes about 7 wt. % of propane or more, the ethane stream 116 can be mixed with the hydrocracking product stream 108, such that the mixture of the ethane stream 116 and the hydrocracking product stream 108 has a propane content of less than about 7 wt. %. For example, in cases where the hydrocracking product stream 108 includes about 4 wt. % of butane or more, the ethane stream 116 can be mixed with the hydrocracking product stream 108, such that the mixture of the ethane stream 116 and the hydrocracking product stream 108 has a butane content of less than about 4 wt. %. The addition of the ethane stream 116 to the hydrocracking product stream 108 may not be necessary, especially in cases where the propane content of the hydrocracking product stream 108 is already less than about 7 wt. % and the butane content of the hydrocracking product stream 108 is already less than about 4 wt. %. For example, the addition of the ethane stream 116 to the hydrocracking product stream 108 may not be necessary in implementations of the system 100 or 200 that include the de-ethanizer column, since a majority of the propane and butane will have been separated from the ethane by the de-ethanizer column. In some implementations, as shown in FIG. 2, hydrogen 117 is separated (for example, by distillation or membrane separation) from the ethylene product stream 110 and recycled to the hydrocracking unit 102 and/or the hydrotreater in the purification unit 114.

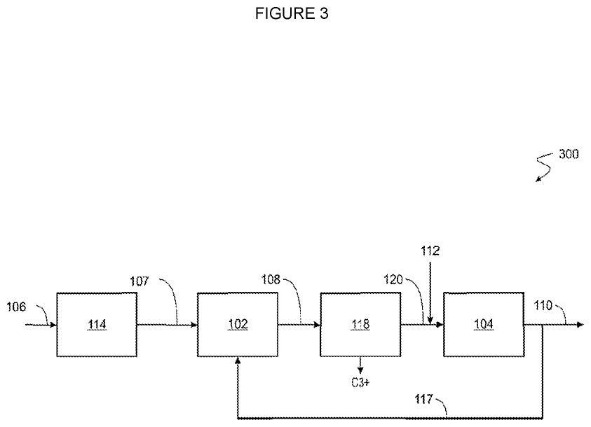

FIG. 3 depicts an example system 300 for producing ethylene that includes ethane separation prior to steam cracking. The system 300 can be substantially similar to the systems 100 and 200 shown in FIGS. 1 and 2, respectively. In addition to the components described with respect to system 100, system 300 can also include a separation unit 118 downstream of the hydrocracking unit 102 and upstream of the steam cracking unit 104. The separation unit 118 is configured to receive the hydrocracking product stream 108 from the hydrocracking unit 102 and separate ethane from heavier hydrocarbons (C2+), such as propane and butane, to produce an ethane stream 120. The ethane stream 120 flows from the separation unit 118 to the steam cracking unit 104. The ethane stream 120 is diluted with steam 112 prior to cracking by the steam cracking unit 104. In some implementations, the separation unit 118 includes a de-ethanizer column. An example of a distillation column which can be implemented as a de-ethanizer column is shown in FIG. 5 and described in more detail later. In some implementations, the separation unit 118 includes pressure swing adsorption (PSA), a membrane separator, or both. In some implementations, as shown in FIG. 3, hydrogen 117 is separated (for example, by distillation or membrane separation) from the ethylene product stream 110 and recycled to the hydrocracking unit 102 and/or the hydrotreater in the purification unit 114.

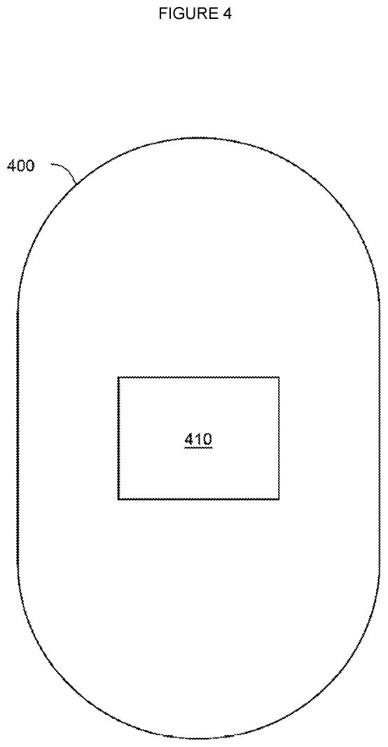

FIG. 4 is a schematic diagram of an example reactor 400 including a catalyst 410. The reactor 400 can be a pressure vessel. The catalyst 410 is disposed within the reactor 400. For example, the reactor 400 is a packed bed reactor, and the catalyst 410 is a packing that fills a packed section of the reactor 400. In some implementations, the packed section of the reactor 400 is filled with random packing of the catalyst 410. In some implementations, the packed section of the reactor 400 is filled with structured packing sections of the catalyst 410, which can be arranged or stacked in different configurations. The catalyst 410 can be selected based on the desired reactions to occur within the reactor 400. The catalyst 410 can be, for example, a hydrocracking catalyst, such that the reactor 400 including the catalyst 410 can be implemented as a hydrocracking reactor in the hydrocracking unit 102. The catalyst 410 can be, for example, a hydrotreatment catalyst, such that the reactor 400 including the catalyst 410 can be implemented as a hydrotreater in the purification unit 114.

FIG. 5 is a schematic diagram of an example distillation column 500. The distillation column 500 receives and fractionates a fluid stream 502 into one or more distillate streams and one or more bottoms streams. In general, a distillate stream has a lower boiling point in comparison to a bottoms stream, and the bottoms stream tends to be heavier (that is, have a higher density) than the distillate stream. For simplicity, the implementation of the distillation column 500 shown in FIG. 5 is configured to produce one distillate stream 512 and one bottoms stream 514. The distillation column 500 includes trays 504, a reboiler 506, a condenser 508, and a reflux drum 510. The reboiler 506 provides heat to the bottom of the distillation column 500, while the condenser 508 removes heat from the top of the distillation column 500. Thus, each of the trays 504 operate at different equilibrium temperatures that decrease in the upward direction along the distillation column 500. The reflux drum 510 serves as an accumulator for distributing the distillate as product and reflux back to the distillation column 500. In cases where reflux is not provided back to the distillation column 500, the reflux drum 510 may be omitted. In cases where the distillate stream 512 is to be transported from the distillation column 500 as a vapor stream, the condenser 508 may be omitted. The distillation column 500 can be, for example, implemented as a de-methanizer, in which the distillate stream 512 includes methane and components lighter than methane (such as hydrogen), and the bottoms stream 514 includes components heavier than methane, such as C1+ alkanes (for example, ethane, propane, and butane). The distillation column 500 can be, for example, implemented as a de-ethanizer, in which the distillate stream 512 includes ethane, and the bottoms stream 514 includes components heavier than ethane, such as C2+ alkanes (for example, propane and butane). The distillation column 500 can be, for example, implemented as a separator to separate hydrogen from ethylene to form the ethylene product stream 110 and to recycle the hydrogen to the hydrocracking unit 102 and/or the hydrotreater in the purification unit 114.

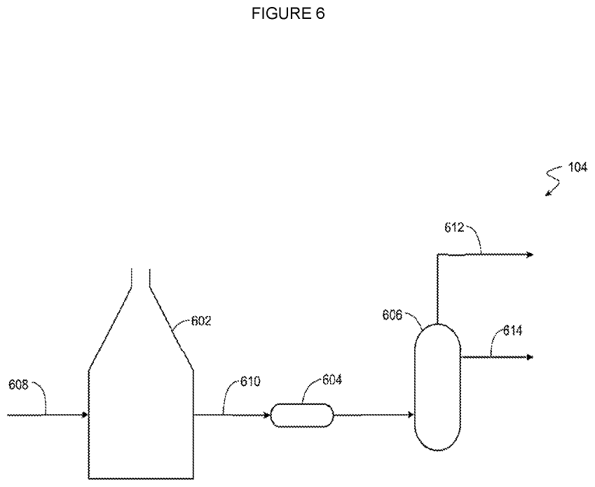

FIG. 6 is a schematic diagram of an example of the steam cracking unit 104. The steam cracking unit 104 includes a furnace 602, a quench unit 604, and a separator 606. A fluid stream 608 that has been diluted with steam (for example, the hydrocracking product stream 108 diluted with steam 112) enters the furnace 602. Steam cracking involves the cracking of alkanes into alkenes. The conversion of alkanes into alkenes produces hydrogen gas. The furnace 602 heats the fluid stream 608 quickly to a temperature in a range of from about 700° C. to about 1,000° C., from about 800° C. to about 900° C., or from about 800° C. to about 850° C. to form a cracked stream 610 that includes one or more alkenes, such as ethylene. In some implementations, the residence time of the fluid stream 608 through the furnace 602 is less than about 1.5 seconds or less than about 1 second. For example, the residence time of the fluid stream 608 through the furnace 602 can be a few milliseconds. The quench unit 604 quickly cools the cracked stream 610 to a temperature less than about 500° C., less than about 450° C., less than about 400° C., less than about 350° C., or less than about 300° C. In some implementations, the quench unit 604 includes a transfer-line heat exchanger. The separator 606 separates hydrogen from the alkenes produced from the steam cracking to form a hydrogen stream 612 and an alkene stream 614. The alkene stream 614 includes the one or more alkenes produced by the steam cracking in the furnace 602. For example, the alkene stream 614 is the ethylene product stream 110 that includes at least 99 wt. % (for example, at least 99.5 wt. %, at least 99.9 wt. %, or at least 99.99 wt. %) ethylene. In some implementations, the separator 606 is a distillation column (for example, an implementation of the distillation column 500). In some implementations, the steam cracking unit 104 includes a compressor (not shown) downstream of the quench unit 604 and upstream of the separator 606. The hydrogen stream 612 can, for example, be recycled to the hydrocracking unit 102 and/or the hydrotreater in the purification unit 114.

Although not shown in FIGS. 1, 2, 3, 4, 5, and 6, the systems (100, 200, 300), the reactor 400, the distillation column 500, and the steam cracking unit 104 can (and are expected to) include the typical components included in similar systems. For example, in each of the configurations described, process streams (also referred to as “streams”) are flowed within each unit and between units of the respective system. The process streams can be flowed using one or more flow control systems implemented throughout the respective system. A flow control system can include one or more pumps to flow the process streams, one or more blowers/compressors to flow the process streams, one or more flow pipes through which the process streams are flowed, and one or more flow elements (such as valves and orifice plates) to regulate the flow of streams through the pipes.

In some implementations, a flow control system can be operated manually. For example, an operator can set a flow rate for each pump by changing the position of a valve (open, partially open, or closed) to regulate the flow of the process streams through the pipes in the flow control system. Once the operator has set the flow rates and the valve positions for all flow control systems distributed across the respective system, the flow control system can flow the streams within a unit or between units under constant flow conditions, for example, constant volumetric or mass flow rates. To change the flow conditions, the operator can manually operate the flow control system, for example, by changing the valve position.

In some implementations, a flow control system can be operated automatically. For example, the flow control system can be connected to a computer system to operate the flow control system. The computer system can include a computer-readable medium storing instructions (such as flow control instructions) executable by one or more processors to perform operations (such as flow control operations). For example, an operator can set the flow rates by setting the valve positions for all flow control systems distributed across the respective system using the computer system. In such implementations, the operator can manually change the flow conditions by providing inputs through the computer system. In such implementations, the computer system can automatically (that is, without manual intervention) control one or more of the flow control systems, for example, using feedback systems implemented in one or more units and connected to the computer system. For example, a sensor (such as a pressure sensor or temperature sensor) can be connected to a pipe through which a process stream flows. The sensor can monitor and provide operating conditions (such as a pressure or temperature) of the process stream to the computer system. In response to the operating condition deviating from a set point (such as a target pressure value or target temperature value) or exceeding a threshold (such as a threshold pressure value or threshold temperature value), the computer system can automatically perform operations to adjust properties of the flow control system. For example, if the pressure or temperature in the pipe exceeds the threshold pressure value or the threshold temperature value, respectively, the computer system can provide a signal to open a valve to relieve pressure or a signal to shut down process stream flow.

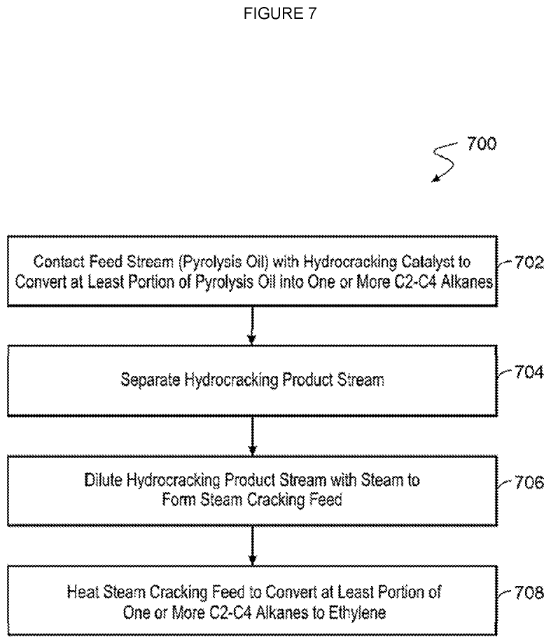

FIG. 7 is a flow chart of an example method 700 for producing ethylene. Any of the systems 100, 200, or 300 can be used, for example, to implement the method 700. At block 702, a feed stream (such as the feed stream 107) is contacted with a hydrocracking catalyst (such as the hydrocracking catalyst disposed within the hydrocracking reactor of the hydrocracking unit 102) in the presence of hydrogen. As mentioned previously, the feed stream 107 includes pyrolysis oil that has been derived from pyrolysis of waste plastic. Contacting the feed stream 107 with the hydrocracking catalyst in the presence of hydrogen at block 702 converts at least a portion of the pyrolysis oil into one or more C2-C4 alkanes to produce a yield in a range of from about 40 wt. % to about 100 wt. %, from about 60 wt. % to about 98 wt. %, or from about 70 wt. % to about 95 wt. % of the one or more C2-C4 alkanes. In some implementations, the method 700 includes purifying the feed stream 106 to form the feed stream 107 prior to block 702. Purifying the feed stream 106 to form the feed stream 107 can include removing sulfur-containing compounds from the feed stream 106. Purifying the feed stream 106 to form the feed stream 107 can include contacting the feed stream 106 with a hydrotreatment catalyst in the presence of hydrogen under hydrotreatment conditions.

At block 704, a hydrocracking product stream (such as the hydrocracking product stream 108) is removed from the hydrocracking unit 102. As mentioned previously, the hydrocracking product stream 108 includes the one or more C2-C4 alkanes produced in the hydrocracking unit 102. In some implementations, removing the hydrocracking product stream 108 from the hydrocracking unit 102 at block 704 includes separating hydrogen and methane from the one or more C2-C4 alkanes, such that the hydrocracking product stream 108 is substantially free of hydrogen and methane. In some implementations, the separated hydrogen, the separated methane, or both are recycled to the hydrocracking reactor of the hydrocracking unit 102. In some implementations, removing the hydrocracking product stream 108 from the hydrocracking unit 102 at block 704 includes separating at least a portion of propane and butane from the one or more C2-C4 alkanes, such that the hydrocracking product stream includes less than about 7 wt. % of propane and less than about 4 wt. % of butane. In some implementations, removing the hydrocracking product stream 108 from the hydrocracking unit 102 at block 704 includes separating C4+ hydrocarbons from the one or more C2-C4 alkanes, such that the hydrocracking product stream 108 is substantially free of C4+ hydrocarbons. In some implementations, at least a portion of the separated C4+ hydrocarbons is recycled to the hydrocracking reactor of the hydrocracking unit 102.

At block 706, the hydrocracking product stream 108 is diluted with steam (such as the steam 112) to form a steam cracking feed stream. At block 708, the steam cracking feed stream is heated in a steam cracker (such as the steam cracking unit 104) to convert at least a portion of the one or more C2-C4 alkanes to ethylene. The ethylene formed at block 708 can be separated to form an ethylene product stream (such as the ethylene product stream 110). Converting the portion of the one or more C2-C4 alkanes to ethylene at block 708 produces hydrogen. In some implementations, at least a portion of the hydrogen formed at block 708 is separated from the ethylene formed at block 708. The hydrogen can then be recycled to the hydrocracking unit 102 and/or the hydrotreater in the purification unit 114.

### Examples

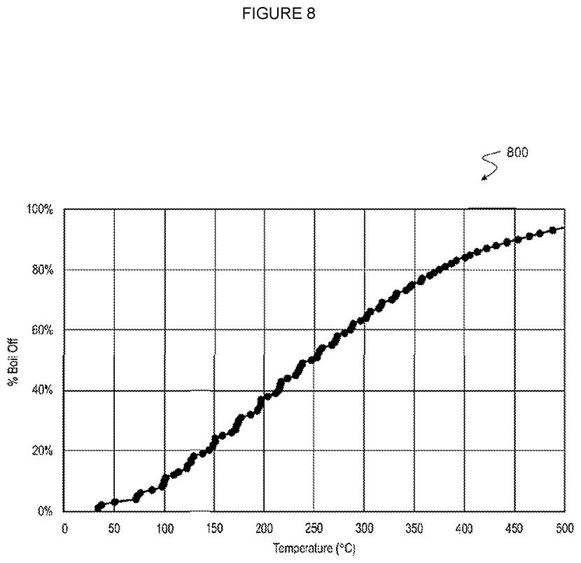

FIG. 8 is a plot 800 of vaporization percent versus temperature (distillation curve) for an example pyrolysis oil that can be implemented as the feed stream 106. The example pyrolysis oil was synthesized in a lab unit that include a 2-liter autoclave loaded with 500 grams of plastic resin. The autoclave was heated to an operating temperature in a range of from 400° C. to 500° C. and held at a pressure of up to 20 psig to thermally decompose the resin over a period of from 6 to 9 hours. The product effluent was removed from the autoclave and cooled to 20° C. by a downstream condenser. The cooled liquid product (pyrolysis oil) was then flowed to a 4-liter knock out pot. The overall yield of pyrolysis oil from the autoclave depended on the feedstock of plastic resin, but for all tests, the pyoil yield was at least 60 wt. % (and in some cases at least 65 wt. %, at least 80 wt. %, or at least 85 wt. %).

The curve shown in plot 800 represents the vaporization behavior of the pyrolysis oil as temperature increases. The pyrolysis oil whose distillation curve is shown by plot 800 in FIG. 8 had the following composition: 25.5% normal paraffins, 7.6% iso-paraffins, 15.6% olefins, 7.0% naphthenes, and 8.7% aromatics. The example pyrolysis oil had a total boiling point (that is, 100% vaporization) of about 600° C.

The example pyrolysis oil underwent a hydrotreatment in two packed bed reactors connected in series. The first packed bed was operated at 350° C., and the second packed bed was operated at 380° C. The hydrotreatment process had the following operating conditions: operating pressure of 6,895 kPag, LHSV of 0.5 per hour, and a volume ratio of hydrogen to the example pyrolysis oil of 2,000:1. The hydrotreatment process ran for a time duration of 60 hours. The hydrotreated pyrolysis oil (an example of the purified feed stream 107) was analyzed. It was found that the hydrotreated pyrolysis oil (purified feed stream 107) included less than 1% of olefins, which signifies that the olefins originally in the pyrolysis oil (unpurified feed stream 106) were hydrogenated by the hydrotreatment process.

The hydrotreated pyrolysis oil (purified feed stream 107) was fed to a hydrocracker that included a packed bed of a hydrocracking catalyst. The hydrocracking process had the following operating conditions: an operating temperature of 400° C., an operating pressure of 5,600 kPag, an LHSV of 1 per hour, and a volume ratio of hydrogen to the hydrotreated pyrolysis oil of 2,000:1.

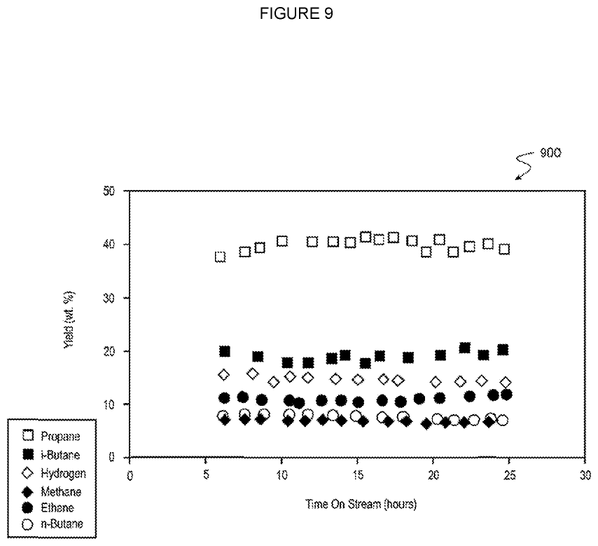

FIG. 9 is a plot 900 of percent yield for various components versus time on stream for the hydrocracking product (an example of the hydrocracking product stream 108). The gaseous portion of the hydrocracking product (hydrocracking product stream 108) was analyzed by gas chromatography, and the liquid portion of the hydrocracking product (hydrocracking product stream 108) was analyzed by liquid chromatography. It was found that the about 90% of the hydrotreated pyrolysis oil (purified feed stream 107) was converted to C2-C4 alkanes (that is, ethane, propane, and butane). The hydrocracking product (hydrocracking product stream 108) included an average yield of about 8 wt. % ethane.

Table 1 provides a gas product composition of the example hydrocracking product (hydrocracking product stream 108). The example hydrocracking product (hydrocracking product stream 108) had an average molecular weight of 9.98 grams per mole. As shown in Table 1, over 90% of the carbon-based components (hydrocarbons, excluding hydrogen gas) were C2-C4 alkanes.7.

TABLE 1
Gas Composition of Hydrocracking Product

| | Concentration | Yield |
|---|---|---|
| Component | (wt. %) | (wt. %) |
| Hydrogen (H₂) | 15.8 | N/A |
| Methane (CH₄) | 6.8 | 8.1 |
| Ethylene (C₂H₄) | 0.1 | Negligible |
| Ethane (C₂H₆) | 6.9 | 6.9 |
| Propane (C₃H₈) | 42 | 45.1 |
| Isobutane (i-C₄) | 12.9 | 38.7* |
| Normal Butane (n-C₄) | 14.3 | |
| Isopentane (i-C₅) | 0.9 | 1.4* |
| Normal Pentane (n-C₅) | 0.3 | |

*C4 and C5 isomer contents are combined for yield values.

The hydrocracking product (hydrocracking product stream 108) underwent steam cracking to produce an ethylene product (an example of the ethylene product stream 110 prior to processing to remove other components, such as hydrogen and components heavier than ethylene). Table 2 provides the composition of the example ethylene product (ethylene product stream 110). As shown in Table 2, the ethylene yield was greater than 50%.

TABLE 2
Composition of Steam Cracked Ethylene Product

| Component | Yield (kg/kg) |
|---|---|
| Hydrogen | 0.037678533 |
| Methane | 0.067611596 |
| Acetylene | 0.003298002 |
| Ethylene | 0.504797062 |
| Ethane | 0.324617104 |
| Methyl-Acetylene/Propadiene | 0.000289106 |
| Propylene | 0.015462053 |
| Propane | 0.003970382 |
| N-Butane | 0.003510739 |
| Iso-Butene | 0.000032933 |
| 1-Butene or N-Butene | 0.001134602 |
| 2-Butene (cis & trans) | 0.000860277 |
| Butadiene | 0.013361469 |
| Benzene | 0.010367737 |
| Toluene | 0.001322422 |
| Styrene (vinyl-benzene) | 0.001090976 |
| 1-Pentene | 0.000294159 |
| Naphthalene | 0.000992153 |
| Pentadienes | 0.000302832 |
| Cyclopentadiene | 0.002863873 |
| Cyclo-pentene | 0.001848437 |
| Cyclohexene | 0.000357096 |

### Definitions

Pyrolysis oil (pyoil): An organic oil derived as a byproduct of pyrolysis, steam cracking, and/or crude oil purification, in which its aromatics content is less than 40 wt. %. Pyrolysis oil derived from waste plastic can have a boiling point range of from 15° C. to 600° C. Pyrolysis oil derived from waste plastic can include carbon-containing compounds with a carbon atom count ranging from C5 to C55. Raw, non-purified pyrolysis oil can include a C5-C15 hydrocarbon content that includes from about 20 wt. % to about 40 wt. % paraffins, up to about 50 wt. % olefins, up to about 40 wt. % naphthenes, and up to about 40 wt. % aromatics. Raw, non-purified pyrolysis oil can include a C15+ hydrocarbon content in a range of from 0% (considered as a light pyoil) to about 50% (considered as a heavy/waxy pyoil). Raw pyrolysis oil can be purified to convert at least about 80% of its olefin content into naphthenes and/or paraffins.

Pygas: An organic oil derived as a byproduct of pyrolysis, steam cracking, and/or crude oil purification, in which its aromatics content is over 60 wt. %.

Aromatic compound (aromatic): A chemical compound that includes a conjugated planar ring accompanied by delocalized pi-electron clouds in place of individual alternating double and single bonds.

LPG (C2-C4 alkanes): Liquefied petroleum gas, which is a mixture of alkanes containing 2 to 4 carbon atoms.

Hydrocracking: A catalytic process in which organic molecules are broken into lighter organic molecules by reaction with hydrogen gas.

LHSV: Liquid hourly space velocity, which is the volumetric flow rate (per hour) of liquid feed entering a catalytic reactor per volume of catalyst in the catalytic reactor.

Residence time: Inverse of LHSV (1/LHSV), which is the total time a fluid parcel has spent inside a control volume, such as a reactor.

Process conditions: Operating pressure and temperature (for example, reactor pressure and reactor temperature).

Overall conversion: A ratio of an amount of feed that has reacted to the initial amount of feed.

$$Conversion = \frac{\begin{matrix}Feed pyrolysis oil liquid mass flow rate inlet - \\ Product hydrocarbon condensable liquid mass flow rate outlet\end{matrix}}{Feed pyrolysis oil liquid mass flow rate inlet} \times 100 \%$$

### Yield of Component A:

$${Yield}_{A} = \frac{Mass flow rate of Component A in Gas product}{\begin{matrix}Gas product mass flow rate - \\ Mass flow rate of hydrogen ( {H}_{2} ) in Gas product \times 100 \%\end{matrix}}$$

LPG steam cracking: Thermal cracking of LPG in the presence of steam.

While this specification contains many specific implementation details, these should not be construed as limitations on the scope of what may be claimed, but rather as descriptions of features that may be specific to particular implementations. Certain features that are described in this specification in the context of separate implementations can also be implemented, in combination, in a single implementation. Conversely, various features that are described in the context of a single implementation can also be implemented in multiple implementations, separately, or in any sub-combination. Moreover, although previously described features may be described as acting in certain combinations and even initially claimed as such, one or more features from a claimed combination can, in some cases, be excised from the combination, and the claimed combination may be directed to a sub-combination or variation of a sub-combination.

As used in this disclosure, the terms “a”, “an”, or “the” are used to include one or more than one unless the context clearly dictates otherwise. The term “or” is used to refer to a nonexclusive “or” unless otherwise indicated. The statement “at least one of A and B” has the same meaning as “A, B, or A and B”. In addition, it is to be understood that the phraseology or terminology employed in this disclosure, and not otherwise defined, is for the purpose of description only and not of limitation. Any use of section headings is intended to aid reading of the document and is not to be interpreted as limiting; information that is relevant to a section heading may occur within or outside of that particular section.

As used in this disclosure, the term “about” or “approximately” can allow for a degree of variability in a value or range, for example, within 10%, within 5%, or within 1% of a stated value or of a stated limit of a range.

As used in this disclosure, the term “substantially” refers to a majority of, or mostly, as in at least about 50%, 60%, 70%, 80%, 90%, 95%, 96%, 97%, 98%, 99%, 99.5%, 99.9%, 99.99%, or at least about 99.999% or more.

Values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “0.1% to about 5%” or “0.1% to 5%” should be interpreted to include about 0.1% to about 5%, as well as the individual values (for example, 1%, 2%, 3%, and 4%) and the sub-ranges (for example, 0.1% to 0.5%, 1.1% to 2.2%, 3.3% to 4.4%) within the indicated range. The statement “X to Y” has the same meaning as “about X to about Y”, unless indicated otherwise. Likewise, the statement “X, Y, or Z” has the same meaning as “about X, about Y, or about Z”, unless indicated otherwise.

Particular implementations of the subject matter have been described. Other implementations, alterations, and permutations of the described implementations are within the scope of the following claims as will be apparent to those skilled in the art. While operations are depicted in the drawings or claims in a particular order, this should not be understood as requiring that such operations be performed in the particular order shown or in sequential order, or that all illustrated operations be performed (some operations may be considered optional), to achieve desirable results. In certain circumstances, multitasking or parallel processing (or a combination of multitasking and parallel processing) may be advantageous and performed as deemed appropriate.

Moreover, the separation or integration of various system modules and components in the previously described implementations should not be understood as requiring such separation or integration in all implementations, and it should be understood that the described components and systems can generally be integrated together or packaged into multiple products.

Accordingly, the previously described example implementations do not define or constrain the present disclosure. Other changes, substitutions, and alterations are also possible without departing from the spirit and scope of the present disclosure.

1. A process for producing ethylene, the process comprising:
contacting a feed stream comprising pyrolysis oil with a hydrocracking catalyst disposed within a hydrocracking unit to convert, in the presence of hydrogen, at least a portion of the pyrolysis oil into one or more C2-C4 alkanes to produce a yield in a range of from about 40 wt. % to about 100 wt. % of the one or more C2-C4 alkanes;
removing a hydrocracking product stream from the hydrocracking unit, the hydrocracking product stream comprising the one or more C2-C4 alkanes;
diluting the hydrocracking product stream with steam to form a steam cracking feed stream; and
heating the steam cracking feed stream in a steam cracker to convert at least a portion of the one or more C2-C4 alkanes to ethylene.

2. The process of claim 1, wherein the pyrolysis oil is derived from pyrolysis of waste plastic.

3. The process of claim 1, wherein the hydrocracking product stream comprises at least 90 wt. % of the one or more C2-C4 alkanes.

4. The process of claim 1, wherein the hydrocracking product stream comprises at least 95 wt. % of the one or more C2-C4 alkanes.

5. The process of claim 1, wherein the pyrolysis oil comprises:
from about 20 wt. % to about 40 wt. % of one or more linear alkanes;
from 0 wt. % to about 40 wt. % of one or more cyclic alkanes;
from 0 wt. % to about 50 wt. % of one or more alkenes;
from 0 wt. % to about 40 wt. % of one or more aromatic compounds; and
from 0 wt. % to about 50 wt. % of one or more C15+ hydrocarbons.

6. The process of claim 1, wherein the hydrocracking catalyst comprises a natural zeolite, a synthetic zeolite, bauxite, an alkali oxide, an alkaline metal earth oxide, an aluminum phosphate, a transition metal oxide, or any combination thereof.

7. The process of claim 1, wherein the hydrocracking catalyst comprises palladium dispersed on a zeolite support.

8. The process of claim 1, wherein converting the portion of the one or more C2-C4 alkanes to ethylene produces hydrogen, and the process further comprises separating at least a portion of the hydrogen from the ethylene and recycling the separated portion of the hydrogen to the hydrocracking unit.

9. The process of claim 1, wherein removing the hydrocracking product stream from the hydrocracking unit comprises separating hydrogen and methane from the one or more C2-C4 alkanes, such that the hydrocracking product stream is substantially free of hydrogen and methane, and the process further comprises recycling at least one of the separated hydrogen or the separated methane to the hydrocracking unit.

10. The process of claim 9, wherein removing the hydrocracking product stream from the hydrocracking unit comprises separating C4+ hydrocarbons from the one or more C2-C4 alkanes, such that the hydrocracking product stream is substantially free of C4+ hydrocarbons, and the process further comprises recycling at least a portion of the separated C4+ hydrocarbons to the hydrocracking unit.

11. The process of claim 9, wherein removing the hydrocracking product stream from the hydrocracking unit further comprises separating at least a portion of propane and butane from the one or more C2-C4 alkanes, such that the hydrocracking product stream comprises less than about 7 wt. % of propane and less than about 4 wt. % of butane.

12. The process of claim 9, further comprising adding ethane to the hydrocracking product stream prior to dilution with steam, such that the hydrocracking product stream comprises less than about 7 wt. % of propane and less than about 4 wt. % of butane.

13. The process of claim 1, further comprising purifying the feed stream to remove heteroatom-containing compounds, metal-containing compounds, or any combinations thereof from the feed stream prior to contacting the feed stream with the hydrocracking catalyst in the presence of hydrogen.

14. The process of claim 13, wherein purifying the feed stream comprises contacting the feed stream with a hydrotreatment catalyst in the presence of hydrogen under hydrotreatment conditions and saturating at least a portion of olefins present in the feed stream into naphthenes, paraffins, or any combinations thereof.

15. A system for producing ethylene, the system comprising:
a feed stream comprising pyrolysis oil;
a hydrocracking unit comprising a hydrocracking catalyst configured to convert, in the presence of hydrogen, at least a portion of the pyrolysis oil into one or more C2-C4 alkanes in response to contacting the feed stream to produce a yield in a range of from about 40 wt. % to about 100 wt. % of the one or more C2-C4 alkanes;
a hydrocracking product stream from the hydrocracking unit, the hydrocracking product stream comprising the one or more C2-C4 alkanes; and
a steam cracker configured to receive and heat at least a portion of the hydrocracking product stream diluted with steam to convert at least a portion of the one or more C2-C4 alkanes to ethylene.

16. The system of claim 15, wherein the pyrolysis oil is derived from pyrolysis of waste plastic.

17. The system of claim 15, wherein the hydrocracking product stream comprises at least 90 wt. % of the one or more C2-C4 alkanes.

18. The system of claim 15, wherein the hydrocracking product stream comprises at least 95 wt. % of the one or more C2-C4 alkanes.

19. The system of claim 15, wherein the pyrolysis oil comprises:
from about 20 wt. % to about 40 wt. % of one or more linear alkanes;
from 0 wt. % to about 40 wt. % of one or more cyclic alkanes;
from 0 wt. % to about 50 wt. % of one or more alkenes;
from 0 wt. % to about 40 wt. % of one or more aromatic compounds; and
from 0 wt. % to about 50 wt. % of one or more C15+ hydrocarbons.

20. The system of claim 15, wherein the hydrocracking catalyst comprises a natural zeolite, a synthetic zeolite, bauxite, an alkali oxide, an alkaline metal earth oxide, an aluminum phosphate, a transition metal oxide, or any combination thereof.

21. The system of claim 15, wherein the hydrocracking catalyst comprises palladium dispersed on a zeolite support.

22. The system of claim 15, further comprising a de-methanizer column configured to receive the hydrocracking product stream and separate hydrogen and methane from the one or more C2-C4 alkanes, such that the hydrocracking product stream is substantially free of hydrogen and methane, and the hydrocracking unit is configured to receive at least one of the separated hydrogen or the separated methane.

23. The system of claim 22, further comprising a separator configured to receive the hydrocracking product stream and separate C4+ hydrocarbons from the one or more C2-C4 alkanes, such that the hydrocracking product stream is substantially free of C4+ hydrocarbons, and the hydrocracking unit is configured to receive at least a portion of the separated C4+ hydrocarbons.

24. The system of claim 22, further comprising a de-ethanizer column configured to receive the hydrocracking product stream and remove ethane to form an ethane stream, and the ethane stream is diluted with steam and heated by the steam cracker.

25. The system of claim 22, further comprising an ethane stream that mixes with the hydrocracking product stream upstream of the steam cracker, such that the portion of the hydrocracking product stream that is diluted with steam and heated by the steam cracker comprises less than about 7 wt. % of propane and less than about 4 wt. % of butane.

26. The system of claim 15, further comprising a purification unit upstream of the hydrocracking unit, the purification unit configured to remove heteroatom-containing compounds, metal-containing compounds, or any combinations thereof from the feed stream.

27. The system of claim 26, wherein the purification unit comprises a hydrotreater comprising a hydrotreatment catalyst configured to, in response to contacting the feed stream in the presence of hydrogen under hydrotreatment conditions, saturate at least a portion of olefins present in the feed stream into naphthenes, paraffins, or any combinations thereof.
